(19) United States
(12) Patent Application Publication
(10) Pub. No.: US 2012/0271384 A1
(43) Pub. Date: Oct. 25, 2012

(54) Device for Preventing or Ameliorating Seasonal Affective Disorder

(76) Inventor: Michael Mark Muehlemann, Liverpool, NY (US)

(21) Appl. No.: 13/251,697

(22) Filed: Oct. 3, 2011

Related U.S. Application Data
(60) Provisional application No. 61/389,221, filed on Oct. 2, 2010.

(51) Int. Cl. A61N 5/06

(52) U.S. Cl. 607/90

(57) ABSTRACT

An array of blue-emitting LEDs centered at about 460 nm, with a gaussian distribution of plus-or-minus 20 nm. are housed in an elongated housing and powered from a standard USB cable or equivalent DC power source. The user can select between a higher and lower intensity. The blue wavelengths in this range are very effective for cirtopic receptors of the eye, with little impact upon visual receptors (i.e., rod cells and cone cells). This allows optimal therapeutic effect with minimal visual side effects.

Applicant claims priority under 35 U.S.C. 119(e) of provisional application Ser. No. 61/389,221, filed Oct. 2, 2010.

### BACKGROUND

The National Institute of Health (NIH) estimates that an estimated 36 million Americans suffer from Seasonal Affective Disorder (SAD), a seasonal depressive disorder brought on by a significant reduction in the amount of light impinging on the retina during winter months. Another 40 million suffer from sleep disorders, most of which originate from circadian rhythm disorders. Seasonal affective disorder is rare in those living within 30 degrees of the equator, where daylight hours are long, constant, and extremely bright. The farther one lives from the equator, the more likely they will develop SAD. In northern latitudes, and/or where cloudy conditions persist, significant percentages of the population experience these effects and have been diagnosed with this condition.

Seasonal Affective Disorder was first discovered in the early 1980s by the NIH, and has since been linked to a hormone imbalance in the brain. This hormone imbalance causes sufferers to have any number of symptoms including depression, a general loss of energy, anxiety, difficulty concentrating, loss of libido, increased cravings for carbohydrates and sweets, and a variety of sleep problems.

According to the American Psychiatric Association, Seasonal Affective Disorder is caused primarily by a biochemical imbalance of two hormones called serotonin and melatonin. Without a sufficient amount of light, the brain does not produce enough serotonin, which results in the symptoms of depression. At the same time the brain overproduces melatonin, sometimes referred to as the ‘hibernation hormone’ and it tends to produce this hormone at the wrong time of the day.

Over the past decade, the best and most specific treatment for SAD has been the substantial and repeated early-morning exposure to bright light. Early clinical research at the NIH demonstrated that specialized ‘full spectrum’ white light therapy provided safe and effective treatment for reversing the effects of SAD.

A great deal of circadian research has been performed since then, focusing specifically on the physiological effects that light therapy has on depression. These scientific studies show that light therapy causes an antidepressant response in three measurable chemical ways:

1) Light therapy increases serotonin levels in the brain. Extended periods of darkness, and/or overcast days cause serotonin to be depleted.

2) Bright light suppresses the hormone melatonin. Melatonin is released in the evening time as a signal for the body to withdraw and prepare for sleep. Melatonin is converted from serotonin, and so it lowers available serotonin. Melatonin is important as a nighttime hormone but high melatonin levels during the daytime, or too much melatonin at any time, can cause mood problems. By suppressing melatonin during the daytime, energy levels are increased, activity cycles are extended, and nighttime sleep patterns are improved.

3) Light therapy regulates the Suprachaismatic nucleus (body clock or circadian rhythm). This is a complex chemical regulatory network, of which melatonin and serotonin are active participants. This system regulates our daily activity cycles, and it needs bright light signals to reset itself each day. When it does not receive the appropriate signals, especially for extended periods, it can malfunction.

Light therapy is a safe and natural way to return to the natural biological cycles that our bodies have evolved to emulate. For tens of thousands of years, humans in the temperate climate zones have been active during the daylight hours and restful at night. They have worked long hours during the light of the summer months, and been more sedentary hibernators during the dark winter months.

But in modern times, few of us work outdoors near the equator on a daily basis, and most of us work a set number of hours each day—independent of the time of year. Our indoor environments are significantly dimmer throughout the day, and the spectrum of indoor light is quite different from that found outdoors. Many people now live quite removed from the equator, where the amount of available sunlight and blue sky during the winter months is severely hampered by either very short days and/or significant cloud cover for extended periods of time. All of these changes in the modern world affect a host of complex regulatory networks in the body that have been generically described as our ‘circadian rhythm’.

Numerous clinical studies have shown that when our circadian rhythm is in sync with natural cycles, we sleep well, we eat more regularly, and we experience higher level of emotional stability. Illness, night shift work, jet lag, and winter light deprivation can all contribute to disrupting our Circadian rhythm, which can cause symptoms as simple as daytime drowsiness to more serious cases of insomnia and even depression.

Over the past two decades hundreds of clinical, placebo-controlled studies have been run using light therapy to treat depression. These studies confirm that light therapy is not only as effective as all other methods including medication, but causes no long-term side effects. Additionally, people generally respond within a week or so to light therapy, as compared to several weeks with most medications. And most medications require several trial regimens before an effective prescription can be identified. The first demonstration of clinical effect was at the National Institute of Mental Health in the early 1980's. Soon after, several research centers initiated clinical trials, and more than 2,000 SAD patients have been studied to date. The method has also been used in private practices, mostly by psychiatrists, but also by family doctors and psychologists. The number of clinicians offering light therapy has increased dramatically in the last decade, and most mental health professionals now consider light therapy to be standard treatment for seasonal affective disorder. Researchers at more than 15 medical centers and clinics in both the U.S. and abroad have had much success with light therapy in patients with clear histories of SAD for at least several years.

The reason light therapy is so effective is that it appears to correct some of the root causes of depression. One reason people become depressed is because the brain center that controls the critical hormone cycles malfunctions for some reason. This brain center is called the Suprachaismatic Nucleus and it can easily become imbalanced from a number of stressors including physical trauma, emotional stress, surgery, age, or lack of light (as with Seasonal Affective Disorder).

When the body clock becomes imbalanced, it produces the wrong hormones, at the wrong times, and can cause a host of problems including insomnia, energy, sex drive, and mood problems. In fact, many people who suffer from depression also have a body clock problem, and they tend to suffer sleep problems and/or feel worse at a particular time of day.

Research over the past decade has provided overwhelming evidence that in addition to the standard rods and cones used for vision, all vertebrates have additional receptors in the eye that provide a wide range of functions.

In 2002 the prestigious journal ‘Science’ announced that a breakthrough scientific discovery had been made for SAD patients. A new set of receptors named the intrinsically photosensitive Retinal Ganglion Cells (ipRGCs) were isolated and characterized in great detail, ranking this as one of the 10 most important scientific breakthroughs of the year.

Over the next 5 years, these non-imaging receptors were shown to mediate numerous non-visual phenomena, including the entrainment of the circadian clock to light-dark cycles, the response of the pupil to light levels, and most importantly the regulation of the critical hormones serotonin and melatonin that regulate the complex interrelated functions of sleep, mood and appetite. During this detailed characterization, researchers discovered that these receptors are most sensitive to the blue portion of the light spectrum, and that they tend to be prevalent towards the lower portion of the eyeball, below the visual portion of the retina as shown in FIG. 1. These findings allow one to postulate that perhaps all vertebrates sense the level and amount of blue light just above the horizon (outside the normal visual field of view) for environmental clues as to time of day and season. In fact, researchers at Rensselaer Polytechnic Institute have shown that blue light, positioned above the field of view, provides significantly superior results in clinical environments. The receptors that regulate melatonin and serotonin are separate from those that we use for vision. They are located in the lower portion of the eyeball and respond to blue light from above the horizon.

The receptors work as triggers for a complex set of hormone controls that have been generically called the circadian rhythm. One of the major discoveries in circadian rhythm research is that the body needs different hormones at different times of day. For example, certain hormones such as melatonin are released by the control center of the brain to help us slow down, withdraw, and go to sleep. Other hormones such as serotonin aren't needed at night, and are released during the day.

The control center that regulates these hormones is called the Suprachaismatic Nucleus, or body clock, and is located in the hypothalamus near the back of the brain. The body clock uses signals like sunlight or specialized bright light to know when to produce the active energetic hormones, and it uses darkness to know when to produce the nighttime

hormones. The body clock can easily malfunction from age, trauma, stress or not getting the proper light signals at the right time of each day. There are a number of companies that sell light therapy units. Many of these companies sell ‘full spectrum’ light boxes or panels. Some are now selling units which incorporate blue LEDs. At least one company sells a unit which uses USB power. Configurations include table top panels, desktop lamps, and even baseball caps with illuminated visors.

The Invention

The Syrcadian Blue™ light therapy unit has been designed with the new discovered ipRGC receptors in mind. As seen in the graph below, the Syrcadian Blue™ device provides high intensity photons at precisely the blue wavelengths required (460 nm in the preferred embodiment) to activate these Cirtopic ipRGC receptors. Because the photopic rods and cones used for vision are not very sensitive to these blue wavelengths, the Syrcadian Blue™ can provide maximum therapeutic intensity, while minimizing the normal side effects experienced with full spectrum light therapy including blinding glare, headaches, and even nausea.

These technical factors allow the Syrcadian Blue™ to be manufactured using the latest Light Emitting Diode (LED) technology, providing a very cost effective therapeutic device.

In a preferred embodiment, which can be situated at the top of a computer monitor or screen, a row of blue-emitting LEDs are housed in an elongated housing and are visible through a lens at the front of the housing. The lens can be transparent or translucent (i.e., frosted). The housing is dimensioned so that the row of LEDs is about 13 to 18 cm across, and about 1/2 cm to one cm vertically. This can be powered from a standard USB cable from the computer or from an equivalent DC power source. There is a HI-LO switch on the housing so the user can select between a higher and a lower intensity. This can be 25 microwatts/square cm (at a working distance of 500 mm) for the low setting, and 50 microwatts/square cm for the high setting. The size and intensity of the light can be adjusted as need be for automotive applications, where the blue-emitting device is placed at the level of the top of the automobile windscreen, e.g., on a car visor. For use with a computer monitor, a clamp fits onto the top of the flat screen frame, and can be adjusted front-to-back to secure the device to the frame.

In the preferred embodiment, the LEDs have a peak wavelength of about 460 nm, and a gaussian or gaussian-like wavelength distribution, over plus-or-minus 20 nm. The blue wavelengths in this range are very effective for the cirtopic receptors of the eye, but are less effective for the visual receptors (i.e., rod cells and cone cells). This allows optimal therapeutic effect with minimal visual side effects.

The preferred device, with the intensities discussed above, is designed to provide the maximum therapeutic effect in the minimal amount of time, without danger to the retina or excessive visual side effects or eye strain. The device could be varied to operate at higher or lower irradiance values. Also, the invention is not limited only to devices with their wavelength in the same range as used in this preferred embodiment.

### BRIEF DESCRIPTION OF THE DRAWING

FIG. 1 is a schematic view of the human eye, in cross section, illustrating the receptors that regulate melatonin and serotonin are separate from those that are used for vision. These ganglion cells are located in the lower portion of the eye and respond to blue light from above the horizon.

FIGS. 2 and 3 illustrate the light therapy device of this invention being positioned above a computer monitor screen, and upon the screen of a laptop device, respectively.

FIG. 4 illustrates the light therapy device of this invention being mounted on an automotive visor.

FIG. 5 is a chart illustrating spectral sensitivity for different retinal sensors of the human eye.

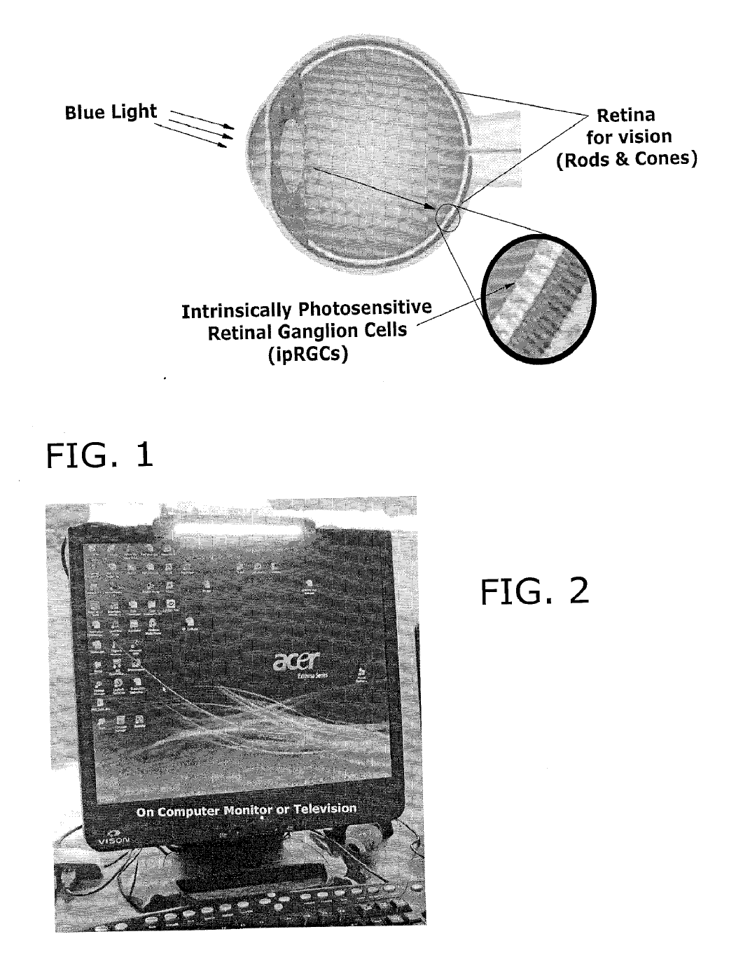

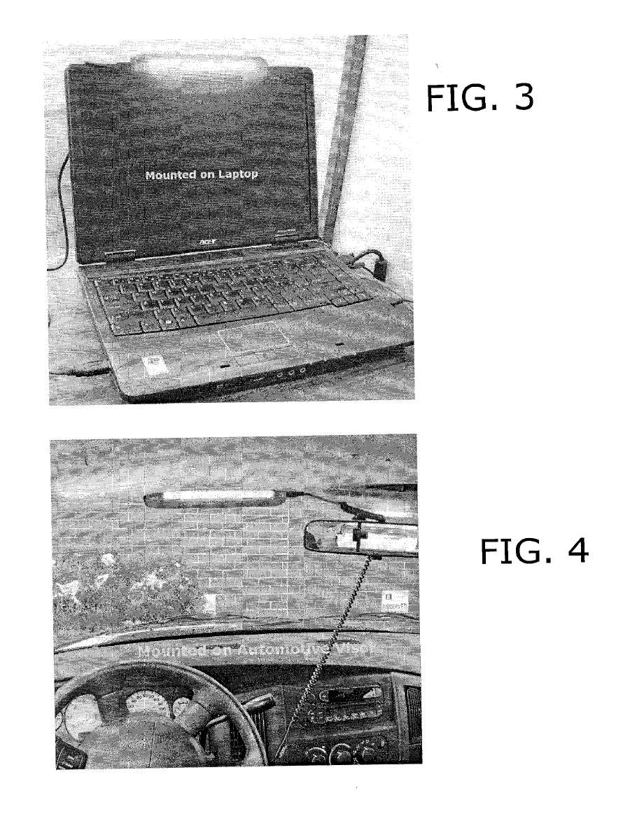

### DETAILED DESCRIPTION

The key to the effective and non-intrusive method of this product are as follows:

1) The Syrcadian Blue™ light therapy device operates as an integral part of your computer monitor or screen, or your television screen (both places that average users spend a considerable amount of time—a necessary condition for light therapy). The position of the device is best placed above your field of view, so that your ipRGCs may be properly illuminated as you scan up and down the screen, similar to the irradiation that would be provided to your retinas on a cloudless day. The therapy is best when it can be applied for more than 90 minutes per day. Since the average person spends 2.9 hours per day at their computer monitor (mostly in the early morning hours), the computer monitor is an ideal integration point for light therapy. While the current product is an aftermarket configuration, the patent concept extends to OEM manufacture and marketing of lighting products that are near or integrated into the computer or television monitor, panel or display screen specifically for the treatment of SAD. These light therapy devices may use blue light, full spectrum light, or some combination of both. The light may be generated by a series of multiple LEDs, a single LED, or by any other light source methods known to one skilled in the art.

2) In a similar manner, the Syrcadian Blue™ light therapy device is also designed to mount in an automobile using an accessory kit (which includes sun visor clips and 12V to USB power converter). The position of the device then once again properly placed above your field of view, so that your ipRGCs may be properly illuminated as you scan down the road, similar to the irradiation that would be provided to your retinas on a cloudless day. This provides a secondary application for individuals with lengthy early morning commutes. While the current product is an aftermarket configuration, the patent concept extends to OEM manufacture and marketing of lighting products which are integrated into or near the automotive visor or windshield area specifically for the treatment of SAD. The light may be generated by a series of multiple LEDs, a single LED, or by any other light source methods known to one skilled in the art.

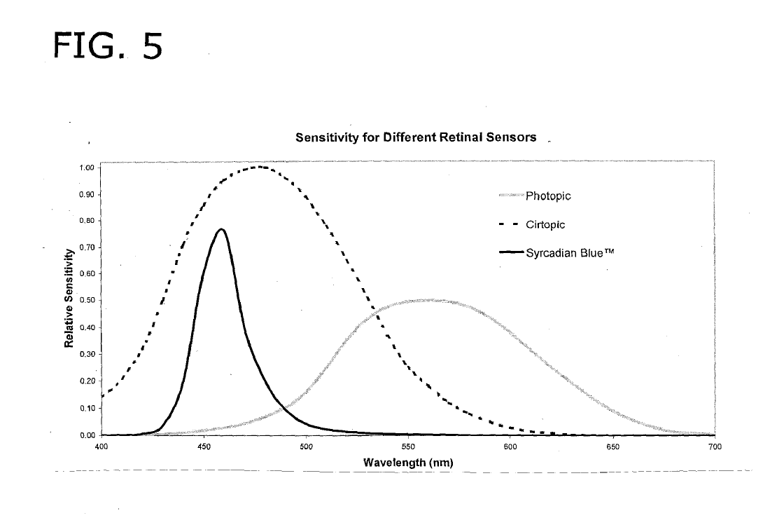

3) In the preferred embodiment, the light therapy device is designed primarily to use only blue wavelengths of light. The wavelength range of light is designed to be predominantly below the photopic response curve, and above the lower sensitivity limit for the cirtopic response curve (as shown in FIG. 5).

4) This preferred range is about 400 nm to 475 nm. In the preferred embodiment the peak wavelength is 460 nm, but could be several wavelengths within this range. This operation provides maximum stimulation of the ipRGCs, with minimum stimulation of the rods and cones used for vision. Because of this configuration, the stimulation of the visual receptors is minimal, and therefore the side effects are dramatically reduced. This leads to dramatically improved patient compliance (the number one reason why light therapy fails). While these LEDs produce significant amounts of blue light, they do so at a precise wavelength where your visual receptors (rods and cones) are only about 9% efficient. That means that the actual therapeutic intensity is about 11 times what it appears visually, maximizing therapeutic activity while eliminating the ‘blinding effects’ of full spectrum light therapy devices. This is the technological secret to being able to provide maximum therapeutic value and minimum visual side effects. The Syrcadian Blue™ light therapy device incorporates a soft start feature that gradually allows your pupils to adjust to the light levels as the intensity increases (at start up and during any dose setting change). The change in intensity is almost imperceptible to the human eye, but eventually reaches its full value in about 90 seconds. By slowing down the natural pupillary light reflex, the Syrcadian Blue™ further minimizes unpleasant side effects. The Syrcadian Blue™ light therapy device has been designed so that you do not need to stare directly into the light. In fact, you will receive faster therapeutic benefit if you do not stare into the light, but rather keep the light above your field of view.

I claim:

1. Device for preventing or ameliorating seasonal affective disorder (SAD) by providing a sufficient exposure of blue light wavelengths to stimulate intrinsically sensitive retinal ganglion cells which are present on the lower portion of the retina of the human eye, comprising
a blue light emitter;
means for mounting the blue light emitter above a visual work area; and
means providing electrical power to the blue light emitter of suitable voltage and current to power the blue light emitter.

2. The Device of claim 1, wherein said visual work area includes a computer monitor, and said mounting means is positioned at a top portion of said monitor.

3. The Device of claim 1, wherein said visual work area includes a television having a viewing screen, and said mounting means is positioned at a top portion of said viewing screen.

4. The Device of claim 1, wherein said visual work area is a driving compartment of a motor vehicle having a windshield through which a motorist views a roadway, and said mounting means is positioned within said driving compartment at a top portion of said windshield.

5. The Device of claim 1, wherein said blue light emitter provides a nominal irradiance of 25 to 50 microwatts per square centimeter at a distance of about 500 mm.

6. The Device of claim 1, wherein said blue emitter provides illumination at a peak wavelength of about 460 nm in a Gaussian distribution within about ±20 nm.

7. The Device of claim 1, wherein said blue emitter includes an elongated housing; and a row of blue LEDs arranged in said housing and visible through a transparent or translucent window on said housing.

8. The Device of claim 7, wherein said row of blue LEDs is about one-half to one cm in height and about thirteen to eighteen cm in width.
